(19) United States
(12) Patent Application Publication
(10) Pub. No.: US 2026/0083127 A1
(43) Pub. Date: Mar. 26, 2026

(54) Thiolactone-Derived Compounds for Generating Nitric Oxide and Methods of Forming the Same

(71) Applicant: Sterile State, LLC, Farmington Hills, MI (US)

(72) Inventor: Megan Cecelia Frost, Hancock, MI (US)

(73) Assignee: Sterile State, LLC, Farmington Hills, MI (US)

(21) Appl. No.: 19/128,108

(22) PCT Filed: Nov. 20, 2023
(86) PCT No.: PCT/US2023/080533

§ 371 Date: May 7, 2025

Related U.S. Application Data
(60) Provisional application No. 63/427,051, filed on Nov. 21, 2022.

(51) Int. Cl. A01N 43/20; A01P 1/00

(52) U.S. Cl. CPC A01N 43/20; A01P 1/00

(57) ABSTRACT

A nitric oxide precursor for providing nitric oxide is provided. The nitric oxide precursor comprises a reaction product of a thiolactone, a primary amine, and a nitrosating compound. The nitric oxide precursor is capable of decomposing to form nitric oxide.

### CROSS-REFERENCE TO RELATED APPLICATIONS

This application claims priority to US Provisional application with the Ser. No. 63/427,051, which was filed Nov. 21, 2022, and which is incorporated by reference herein.

### FIELD OF THE INVENTION

The present disclosure generally relates to a nitric oxide precursor comprising a reaction product of a thiolactone, a primary amine, and a nitrosating compound.

### BACKGROUND

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

A variety of products and articles, including, for example, medical instruments, devices, and equipment, must be sterilized prior to use to prevent bio-contamination of a wound site, a sample, an organism, or the like. A number of sterilization processes are used which involve contacting the product or article with a sterilant. Examples of such sterilants include dintitrogen tetraoxide, nitric oxide, steam, ethylene oxide, hydrogen peroxide, dry heat, and the like. Conventional methods for forming nitric oxide use catalytic and enzymatic generation of nitric oxide from nitrite or NO donating compounds such as diazeniumdiolates.

For example, in one approach, selected polymers were chemically modified to provide nitrosothiol groups in amounts that are useful for NO based sterilization processes as described in WO 2023/205125. Here, specific polymeric materials were modified in a chemical reaction that introduced pendant nitrosothiol groups into the polymer that then served as reactive groups to anchor a thiol that can be converted to a nitrosothiol group from which nitric oxide can be released. More specifically, polycarbonate-polydimethylsiloxane (PCPDMS) block co-polymer, polyurethane-polydimethylsiloxane (PUPDMS) block co-polymer, polyurethane (PU), poly(ethylene-co-vinyl acetate) copolymer (EVA), or polydimethylsiloxane were first reacted with 3-aminopropyl trimethoxy siloxane to introduce a pendant amine group that was then reacted with acetylpenicillamine thiolactone to generate a pendant thiol group that was subsequently reacted with t-butyl nitrite to convert the pendant thiol group to the corresponding S-nitrosothiol. Unfortunately, such an approach was limited to modified polymers having a relatively high molecular weight and further limited by the specific chemistry required to produce the modified polymers. Still further, such modification required a relatively complex multi-step process.

Thus, such and other methods for forming nitric oxide from a precursor typically require expensive reactants and must be utilized in controlled systems to permit the generation of nitric oxide. Moreover, such nitric oxide precursors have a large molecular weight and as such limit their use in a variety of applications where smaller precursors are required.

Unfortunately, most small molecule nitrosothiols that decompose to form nitric oxide are notoriously unstable, especially when in solution. These limitations have prevented mainstream commercialization of sterilization or sanitation compositions capable of generating nitric oxide by consumers and professionals. Accordingly, there remains a need for improved compositions capable of generating nitric oxide for a variety of medical and consumer purposes.

### BRIEF SUMMARY OF THE INVENTION

A nitric oxide precursor for providing nitric oxide is provided herein. The nitric oxide precursor comprises, consists essentially of, consists of, or is, a reaction product of a thiolactone, a primary amine, and a nitrosating compound. In various embodiments, the thiolactone and the primary amine are capable of reacting to form an intermediate, and the intermediate and the nitrosating compound are capable of reacting to form the nitric oxide precursor. In these and other embodiments, the thiolactone and the primary amine are capable of reacting in the presence of a solvent to form the intermediate. It is contemplated herein that the thiolactone and the primary amine are capable of reacting at a temperature of from about 1° C. to about 25° C.

The nitric oxide precursor is capable of decomposing to form the nitric oxide. In various embodiments, the nitric oxide precursor comprises a nitrosothiol that is capable of decomposing to form the nitric oxide. It is contemplated herein that the nitric oxide precursor is capable of decomposing to form the nitric oxide at a predetermined rate and/or for a predetermined amount of time based on the rate of formation of the nitric oxide precursor resulting from reaction of the intermediate and the nitrosating compound. Furthermore, the nitric oxide precursor may exhibit minimal decomposition to nitric oxide for a time period of at least 5 minutes after forming the nitric oxide precursor, depending on the specific amine compound used and how the precursor is stored. Alternatively, the nitric oxide precursor may exhibit minimal decomposition to nitric oxide for a time period of from 5 minutes to 365 days after forming the nitric oxide precursor, depending on the specific amine compound used and how the precursor is stored. As described in greater detail below, tuning of the rate of formation of the nitric oxide precursor is suitable for a variety of applications requiring such control to form the nitric oxide.

The nitric oxide precursor may be used in a wide variety of medical and consumer applications. The properties of the nitric oxide precursor may be adjusted based on the selection of the primary amine and the nitrosating compound to suit specific applications. Non-limiting examples of suitable adjustments include nitric oxide generation capabilities and the rate of release of nitric oxide. As described in greater detail below, the nitric oxide precursor may be incorporated into a variety of substrates such that nitric oxide is released from the substrate resulting from decomposition of the nitric oxide precursor. In addition to tuning decomposition of the nitric oxide precursor, release of nitric oxide may also be adjusted based on substrate composition and substrate characteristics to further control release of nitric oxide from the substrate. This controlled nitric oxide release is useful for sterilizing and sanitizing medical and consumer devices.

In particular, physiochemical properties of the nitric oxide precursor, such as reactivity, hydrophobicity, number of nitric oxide donors, stability of the oxide donors, etc. can be tuned based on selection of the primary amine. For example, in embodiments where the thiolactone is reacted with a primary amine that also contains a thiol group, the resulting intermediate can be reacted with the nitrosating compound to form a nitric oxide precursor containing two nitrosothiols on one molecule. These nitric oxide precursors can be used as additives for polymer films/matrices, powdered materials, such as desiccants (e.g., silica gel or sodium polyacrylate), or in a solution phase for formulations and applications relating to disinfecting, sanitizing, and sterilizing washes for a wide variety of objects and devices and in a number of applications.

The thiolactone may have a structure according to the following formula (I):

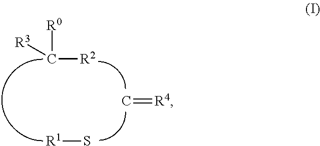

wherein R¹ is a bond or a divalent organic group, R² is a bond or a divalent organic group, and R³ is a hydrogen atom or a monovalent organic group, and R⁴ is an oxygen atom or a sulfur atom. Non-limiting examples of suitable thiolactone groups of the thiolactone include α-acetothiolactone groups, β-propiothiolactone groups, γ-butyrothiolactone groups, δ-valerothiolactone groups, ε-caprothiolactone groups, ζ-enanthothiolactone groups, η-caprylothiolactone groups, and θ-pelargothiolactone groups. In some embodiments, the thiolactone is an amine-containing thiolactone, such as a thietanone (e.g., N-(2,2-Dimethyl-4-oxo-3-thietanyl)acetamide).

In various embodiments, the primary amine comprises a cysteine or derivative thereof, a lysine or derivative thereof, a butylamine or derivative thereof, or a combination thereof. In these and other embodiments, the primary amine contains a thiol-functional group. In exemplary embodiments, the cysteine or derivative thereof comprises cysteine, glutathione, acetyl cysteine, penicillamine, acetylpenicillamine, S-nitroso-n-acetylpenicillamine, bucillamine, or combinations thereof.

In certain embodiments, the nitrosating compound comprises a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnittrite, nitrite salts, ion paired nitrite, silver nitrite, zinc nitrite, iron nitrite, copper nitrite, transition metal-nitrite compounds, or combinations thereof.

Therefore, it should be appreciated that the nitric oxide precursor prepared according to the inventive subject matter has a relatively small molecular weight. Viewed from a different perspective, the nitric oxide precursor will not comprise a polymer (e.g., having four, ten, 20, 100, 1,000 repeat units). For example, contemplated nitric oxide precursors may have a molecular weight of between 100 and 500 Da, or between 250-750 Da, or between 400-1,000 Da, and typically less than 5,000 Da or less than 2,500 Da, or less than 1,500 Da. Unexpectedly, and despite their relatively small molecular weight, the nitric oxide precursors contemplated herein will also have a relatively high stability and can therefore be incorporated into a variety of materials to form a composite article. In some embodiments, the nitric oxide precursor will be in a crystalline form, which may be combined with a carrier or suitable liquid.

The inventors contemplate that the nitric oxide precursor may exhibit decomposition to nitric oxide after forming the reaction product within a predetermined amount of time, such as within 1 hour. Of course, this decomposition of the nitric oxide precursor within a predetermined amount of time may be subject to the minimal decomposition to nitric oxide for a time period of at least 5 minutes after forming the nitric oxide precursor, as described above. In various embodiments, decomposition to nitric oxide may be sustained for a predetermined amount of time, such as a time period of from about 1 minutes to about 1 year. Without being bound by theory, it is believed that properties of the nitric oxide precursor may be adjusted based on the selection of the primary amine and the nitrosating compound for tuning decomposition to nitric oxide suitable for specific applications.

Viewed from a different perspective, the nitric oxide precursor may be incorporated in or with a composite article, such as polymer films and matrices, and powdered materials. Non-limiting examples of suitable powdered materials include desiccants, such as silica gel or sodium polyacrylate desiccants.

A method of sterilizing or sanitizing an article (e.g., a device or object) is also provided herein. The method comprises applying the nitric oxide precursor described above to the article.

In one aspect of the inventive subject matter, the inventor contemplates a method of producing a nitric oxide precursor that includes a step of reacting a thiolactone with a primary amine in a ring-opening reaction in a solvent to form an intermediate with a thiol group and a step of reacting the thiol group of the intermediate with a nitrosating compound in the solvent to thereby form a nitric oxide precursor having a nitrosothiol. In another step, the solvent is at least partially removed. As will be readily appreciated the nitric oxide precursor is capable of decomposing to form nitric oxide.

In some embodiments, the thiolactone has a molecular weight of less than 500 Da. For example, suitable thiolactones will have has a structure according to the following formula (III):

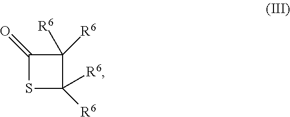

wherein each occurrence of R6 is independently a hydrogen, a hydroxyl, a substituted or unsubstituted C1-C6 alkyl, substituted or unsubstituted C1-C6 heteroalkyl, a substituted or unsubstituted C2-C6 alkenyl, a substituted or unsubstituted C2-C6 herteroalkenyl, a substituted or unsubstituted C1-C6 alkoxy, or a substituted or unsubstituted C1-C6 heteroalkoxy; and wherein when R6 is substituted, the substituent is selected for the group consisting of OH, SH, NH3, NO2, acyl, an amido group, and halogen.

Where desired, the thiolactone comprises an amine group. Therefore, suitable thiolactones include N-(2,2-Dimethyl-4-oxo-3-thietanyl)-acetamide, N-acetylcysteine thiolactone, N-acetyl-homocysteine thiolactone, homocysteine thiolactone, or butyryl-homocysteine thiolactone.

In further embodiments, the primary amine has a molecular weight of less than 500 Da. Among other choices, suitable primary amines include an amino acid. For example, contemplated primary amines include butylamine, cysteine, glutathione, acetyl cysteine, penicillamine, acetylpenicillamine, S-nitroso-n-acetylpenicillamine, and bucillamine.

With respect to nitrosating compounds, it is contemplated that various organonitrite or nitrite salts can be used, and especially contemplated nitrosating compounds include sodium nitrite, calcium nitrite, potassium nitrite, tetrabutylammonium nitrite, dicyclohexylammonium nitrite, butylnitrite, isobutylnitrite, t-butylnitrite, amylnitrite, and pentylnitrite.

Most typically, but not necessarily, the solvent is an aqueous solvent and/or may comprise a polar alcoholic solvent. For example, suitable solvents include water and methanol and/or ethanol. As shown in more detail below, the reaction may be performed in the presence of an acid (e.g., organic acid), and the reaction will advantageously be performed at ambient temperature and ambient pressure. In further contemplated aspects, the solvent may be at least partially (e.g., at least 50%) removed, and/or where desired, the nitric oxide precursor is crystallized. In some embodiments, the nitric oxide precursor exhibits minimal degradation at 23° C. for a time period of at least 1 month. Moreover, it is generally preferred that the nitric oxide precursor does not comprise a polymer having 4 or more repeat units.

Therefore, and viewed from a different perspective, the inventor also contemplates a nitric oxide precursor for providing nitric oxide that comprises a reaction product of a thiolactone, a primary amine, and a nitrosating compound, wherein the nitric oxide precursor is capable of decomposing to form nitric oxide.

In most embodiments, the thiolactone and the primary amine are capable of reacting to form an intermediate, and wherein the intermediate and the nitrosating compound are capable of reacting to form the nitric oxide precursor. Therefore, the thiolactone and the primary amine are capable of reacting in the presence of a solvent to form the intermediate, for example, at a temperature of from about 1° C. to about 25° C. Most typically, the nitric oxide precursor comprises a nitrosothiol that is capable of decomposing to form the nitric oxide. In some embodiments, the nitric oxide precursor exhibits minimal decomposition to nitrogen oxide for at least 24 hours after forming the nitrogen oxide precursor.

It is further contemplated that the thiolactone is an amine-containing thiolactone (e.g., comprising thietanone). Contemplated primary amines may be a cysteine or derivative thereof, a lysine or derivative thereof, a butylamine or derivative thereof, or a combination thereof. Where desired, the primary amine contains a thiol-functional group (e.g., cysteine or derivative thereof, and wherein the cysteine or derivative thereof comprises cysteine, glutathione, acetyl cysteine, penicillamine, acetylpenicillamine, S-nitroso-n-acetylpenicillamine, bucillamine, or combinations thereof).

Suitable nitrosating compounds will include an organic nitrite or a nitrite salt, such as sodium nitrite, calcium nitrite, potassium nitrite, tetrabutylammonium nitrite, dicyclohexylammonium nitrite, butylnitrite, isobutylnitrite, t-butylnitrite, amylnitrite, or pentylnitrite.

In further contemplated aspects, wherein the nitric oxide precursor is in a crystalline form, which will typically exhibit improved storage stability as compared to the corresponding non-crystallized nitric oxide precursor.

In yet further contemplated aspects, of the inventive subject matter, the inventor contemplates a composite article that comprises a carrier a nitric oxide precursor as presented herein. In some embodiments, the composite article exhibits minimal release of nitric oxide for at least 24 hours after forming the nitric oxide precursor. Additionally, the inventor contemplates a method of sterilizing or sanitizing an article in which a nitric oxide precursor as presented herein is applied to the article.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A-1D are images illustrating non-limiting embodiments of the nitric oxide precursor.

FIGS. 2A-2D are images illustrating non-limiting embodiments of the nitric oxide precursor.

FIG. 3A is an image illustrating non-limiting embodiments of the nitric oxide precursor.

FIGS. 3B and 3C are graphs illustrating the release of nitric oxide from non-limiting embodiments of composite articles including the nitric oxide precursor.

FIGS. 4A-4D are images illustrating non-limiting embodiments of composite articles including the nitric oxide precursor.

### DETAILED DESCRIPTION

The following detailed description is merely illustrative in nature and is not intended to limit the embodiments of the subject matter or the application and uses of such embodiments. Furthermore, there is no intention to be bound by any expressed or implied theory presented in the preceding technical field, background, brief summary, or the following detailed description.

A nitric oxide precursor for providing nitric oxide is provided herein. The nitric oxide precursor comprises, consists essentially of, consists of, or is, a reaction product of a thiolactone, a primary amine, and a nitrosating compound. In various embodiments, the thiolactone and the primary amine react to form an intermediate, and the intermediate and the nitrosating compound react to form the nitric oxide precursor. In these and other embodiments, the thiolactone and the primary amine react in the presence of a solvent to form the intermediate. Preferably, but not necessarily, the thiolactone and the primary amine are capable of reacting at a temperature of from about 1° C. to about 25° C. Thus, it should be appreciated that elevated temperatures are not necessary to form the intermediate. An exemplary reaction schematic is shown below:

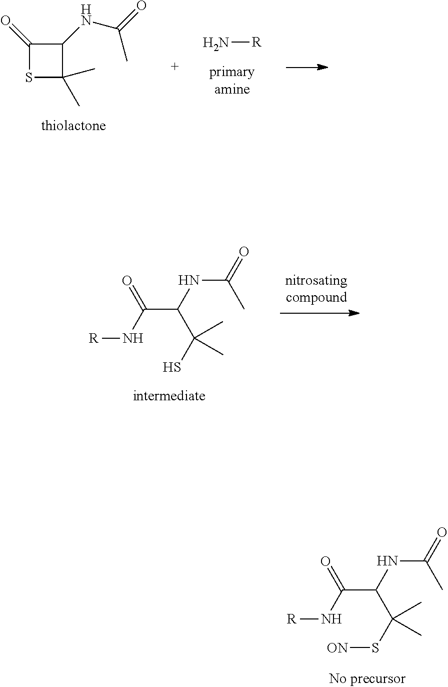

In various embodiments, the nitric oxide precursor comprises a nitrosothiol that is capable of decomposing to form the nitric oxide. Nitric oxide is lipid soluble and has the ability to disrupt the lipid membranes of microorganisms, to modulate cell and tissue response, to control coagulation and biological integration, to impart antimicrobial characteristics, etc. Furthermore, nitric oxide may inactivate thioproteins thereby disrupting the functional proteins of microbes. As will be readily appreciated, nitric oxide can react with ambient air to form various nitrogen oxides such as nitrogen dioxide, dinitrogen oxide, etc. Nitrogen dioxide is more water soluble than nitric oxide. Moreover, nitric oxide and nitrogen dioxide are effective disruptors of DNA, causing strand breaks and other damage leading to an inability for a cell to function.

As used herein, the term “nitric oxide” or “NO” refer to the NO free radical. Nitric oxide can react with ambient air to form various nitrogen oxides, including nitrogen dioxide (NO₂), nitrogen trioxide (NO₃), dinitrogen trioxide (N₂O₃), dinitrogen tetroxide (N₂O₄), dinitrogen pentoxide (N₂O₅) and nitrous oxide (N₂O). As used herein, the phrase “nitric oxide precursor” means a compound or composition capable of producing or releasing nitric oxide.

The nitric oxide precursors contemplated herein may be used in a wide variety of medical and consumer applications. The properties of the nitric oxide precursor may be adjusted based on the selection of the primary amine and the nitrosating compound to suit specific applications. Non-limiting examples of suitable adjustments include nitric oxide generation capabilities and the rate of release of nitric oxide. As described in greater detail below, the nitric oxide precursor may be incorporated into a variety of substrates such that nitric oxide is released from the substrate resulting from decomposition of the nitric oxide precursor. In addition to tuning the rate of decomposition of the nitric oxide precursor to form nitric oxide, release of nitric oxide may also be adjusted based on substrate composition and substrate characteristics to further control release of nitric oxide from the substrate. This controlled nitric oxide release is useful for sterilizing and sanitizing medical and consumer devices.

Viewed from a different perspective, the inventors contemplate utilizing the nitric oxide precursor to form nitric oxide for a variety of medical and consumer applications. In various embodiments, an article (e.g., a device or object) may be treated with the nitric oxide precursor, such as in the form of a liquid, a powder, a film, a coating, or the like. Non-limiting examples of suitable uses of the nitric oxide precursor (e.g., as liquids, powders, films, or coatings) include: detergent or cleaning solutions for sanitizing or sterilizing objects treated with the solution (e.g., sports equipment, such as hockey gloves and cycling gloves, cleaning surgical instruments, internal lumens of endoscopes, surfaces of medical equipment, and the like); detergent or cleaning powders for sanitizing or sterilizing objects treated with the powder hygienic containers for sanitizing hygiene devices (e.g., desiccants and the like); medical device containers for sanitizing medical instruments (e.g., stethoscopes, otoscopes, and the like), medical devices (e.g., portable ultrasound device, communication devices, and the like); components of devices exposed to moisture for resisting growth of mold or mildew (e.g., washing machines, boat compartments, and the like); sporting equipment (e.g., yoga mats, touchable surfaces of strength training equipment, touchable surfaces of cardio equipment, and the like); liners for sporting equipment bags for sanitizing sporting equipment (e.g., shoes, hockey equipment, ski equipment, facemasks, googles, helmets, and the like); food packaging for preserving foodstuff (e.g., meats, fruits, vegetables, cheeses, ingredients thereof, and the like); components of vehicles for sanitizing vehicles (e.g., headliners, seat cushion liners, carpet liners, and the like); and within drawers of cabinets, desks, boxes, etc., to eliminate musty odors.

The inventors contemplate that the nitric oxide precursor may exhibit relatively rapid decomposition to release nitric oxide after forming the nitric oxide precursor within a predetermined amount of time, such as within 1 hour, alternatively within 30 minutes, alternatively within 5 minutes, alternatively within 1 minute, or alternatively within 10 seconds, alternatively within 1 second, or alternatively within 0.1 seconds. Alternatively, the nitric oxide precursor may also be prepared to exhibit relatively minimal decomposition to form nitric oxide after forming the nitric oxide precursor for a time period of at least 5 minutes, alternatively at least 10 minutes, alternatively at least 15 minutes, alternatively at least 30 minutes, alternatively at least 45 minutes, alternatively at least 1 hour, alternatively at least 2 hours, alternatively at least 3 hours, alternatively at least 4 hours, alternatively at least 5 hours, alternatively at least 6, alternatively at least 7 hours, alternatively at least 8 hours, alternatively at least 9 hours, alternatively at least 10 hours, alternatively at least 11 hours, alternatively at least 12, alternatively at least 13 hours, alternatively at least 14 hours, alternatively at least 15 hours, alternatively at least 16 hours, alternatively at least 17 hours, alternatively at least 18 hours, alternatively at least 19 hours, alternatively at least 20 hours, alternatively at least 21 hours, alternatively at least 22 hours, alternatively at least 23 hours, alternatively at least 24 hours, alternatively at least 48 hours, or alternatively at least 365 days, depending on the specific amine compound used and how the precursor is stored.

Therefore, the nitric oxide precursor contemplated herein may exhibit minimal decomposition to nitric oxide for a time period of from 5 minutes to 365 days, alternatively from 5 minutes to 48 hours, alternatively from 5 minutes to 24 hours alternatively from 1 hours to 24 hours, alternatively from 4 hours to 24 hours, alternatively from 8 hours to 24 hours, alternatively from 16 hours to 24 hours, alternatively from 20 hours to 24 hours after forming the nitric oxide precursor, depending on the specific amine compound used and how the precursor is stored. In various embodiments, decomposition of the nitric oxide precursor to form nitric oxide may be sustained for a predetermined amount of time, such as a time period of from about 1 minutes to about 1 year, alternatively from about 1 hour to about 6 months, alternatively from about 24 hours to about 3 months, or alternatively from about 1 week to about 8 weeks. Thus, and viewed from a different perspective, decomposition to form nitric oxide may be sustained for a period of at least 1 minute, alternatively at least 1 hours, alternatively at least 24 hours, alternatively at least 1 week, alternatively at least 4 weeks, alternatively at least 12 weeks, or alternatively at least 26 weeks. Without being bound by theory, it is believed that properties of the nitric oxide precursor may be adjusted based on the selection of the primary amine and the nitrosating compound for tuning decomposition to nitric oxide suitable for specific applications.

In particularly contemplated aspects, the nitric oxide precursor will exhibit desirable storage stability. For example, in various embodiments, the nitric oxide precursor exhibits minimal degradation at 4° C. for a time period of at least 1 month, alternatively at least 2 months, alternatively at least 3 months, alternatively at least 6 months, or alternatively at least 12 months. In other embodiments, the nitric oxide precursor exhibits minimal degradation at 23° C. for a time period of at least 1 month, alternatively at least 2 months, alternatively at least 3 months, alternatively at least 6 months, or alternatively at least 12 months. The phrase “minimal degradation” means that nitric oxide precursor exhibits minimal color difference over a desired time period of 10% of the AE*ab, alternatively 5% of the AE*ab, alternatively 4% of the AE*ab, alternatively 3% of the AE*ab, alternatively 2% of AE*ab, alternatively 1% of the AE*ab, or alternatively 0.1% of the AE*ab in accordance with ASTM D2244-21.

The inventor further contemplates that suitable nitric oxide precursors can be blended into polymers, solution phases, or powders, and used to generate nitric oxide for creating a wide array of NO donating/generating entities by blending the nitric oxide precursor into a carrier (polymer or powder or embedded in and/or on solid carriers) or for creating solutions that generate NO in a controlled and predicable manner from the carrier/solution phase. Herein, a method for synthesizing novel small molecule NO donors is described that can be used for this purpose and formulations and applications of these NO generating moieties that can be used as disinfecting, sanitizing, and sterilizing washes for a wide array of different objects and in a number of different situations.

Furthermore, it should be appreciated that the nitric oxide precursors described herein, and their use in polymers, powders, on solid carriers or as solutions, allows for simple synthesis, controlled formation and release of NO with and without acid (in a polar or non-polar phase). Indeed, the nitric oxide precursors may be formed at ambient conditions and will remain stable over time. Furthermore, the nitric oxide precursors can be crystalized, can endure polymer casting process, can be blended into or otherwise coupled with various different carriers, and can be formed in organic, aqueous, or combined organic and aqueous phases.

In exemplary embodiments, it is contemplated herein that self-protected thiolactones (e.g., formed from N-acetylpenicillamine and the like) are reacted with discrete amine compounds, (e.g., cysteine, leucine, isoleucine, lysine, penicillamine, glutathione, and the like) to form intermediates in the form of monomers, dimers, trimers, tetramers, etc. These small molecule intermediates have the potential of higher loading of NO compounds to create nitric oxide precursors that are tunable. For example, if the thiolactone is reacted with a primary amine that contains a thiol group, the resulting nitric oxide precursor can have two nitrosothiol groups on a single molecule. The combination of various primary amines that contain thiol groups provides a large variety of nitric oxide precursors exhibiting tunable release properties. These nitric oxide precursors can be used as additives to polymer films/matrices, powdered materials such as desiccants (silica gel, or sodium polyacrylate) or in solution phase.

Nitric oxide precursors, such as small molecule nitrosothiols, formed from a thiolactone, a primary amine, and a nitrosating compound may be formed in both organic and aqueous phases. Solutions that contain cleaning agents (e.g., enzymes, surfactants, etc.) may be combined with the nitric oxide precursor. This nitric oxide precursor can then decompose rapidly to generate nitric oxide. These nitric oxide precursors in combination with various carriers, dessicants, and cleaning agents, such as powders or solution components (e.g., silica gel, sodium polyacrylate, Alconox soap, proteases, etc.) may be used for reprocessing articles, such as endoscopes and probes, and sterilizing other objects.

With regard to suitable thiolactones it should be appreciated that there is a wide variety of thiolactones that are appropriate for use herein, depending on the design constraints of the desired application of the nitric oxide precursors. In some embodiments, the thiolactone may have a structure according to the following formula (I):

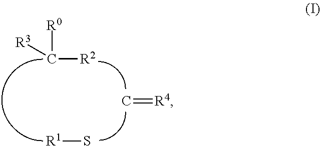

wherein R¹ is a bond or a divalent organic group, R² is a bond or a divalent organic group, and R⁰ and R³ are independently a hydrogen atom or a monovalent organic group, and R⁴ is an oxygen atom or a sulfur atom. Non-limiting examples of suitable thiolactone groups of the thiolactone include α-acetothiolactone groups, β-propiothiolactone groups, γ-butyrothiolactone groups, δ-valerothiolactone groups, ε-caprothiolactone groups, ζ-enanthothiolactone groups, η-caprylothiolactone groups, and θ-pelargothiolactone groups.

For example, contemplated thiolactones may have a structure according to the following formula (II):

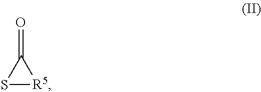

wherein R⁵ is a substituted or unsubstituted C₁-C₁₂ alkyl.

In another example, contemplated thiolactones may have a structure according to the following formula (III):

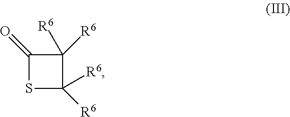

wherein each occurrence of R⁶ is independently a hydrogen, a hydroxyl, a substituted or unsubstituted C₁-C₆ alkyl, substituted or unsubstituted C₁-C₆ heteroalkyl, a substituted or unsubstituted C₂-C₆ alkenyl, a substituted or unsubstituted C₂-C₆ herteroalkenyl, a substituted or unsubstituted C₁-C₆ alkoxy, or a substituted or unsubstituted C₁-C₆ heteroalkoxy.

In yet another example, contemplated thiolactones may have a structure according to the following formula (III):

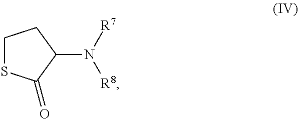

wherein R⁷ is a group comprising an ethylenically unsaturated, polymerizable double bond and R⁸ is selected from the group consisting of H, C₁-C₆ alkyl and C₁-C6 acyl. Alternatively, in formula (IV) R⁷ and R⁸ form together a group comprising an ethylenically unsaturated, polymerizable double bond.

It is further contemplated that the thiolactone may be an amine-containing thiolactone. Non-limiting examples of suitable amine-containing thiolactones include thietanones (e.g., N-(2,2-Dimethyl-4-oxo-3-thietanyl)acetamide), N-acetylcysteine thiolactone, N-acetyl-homocysteine thiolactone, homocysteine thiolactone, butyryl-homocysteine thiolactone, or combinations thereof. In one exemplary embodiments, the amine-containing thiolactone includes N-(2,2-Dimethyl-4-oxo-3-thietanyl)acetamide. In most embodiments, contemplated thiolactones will have a molecular weight of less than 1,000 Da, or less than 900 Da, or less than 800 Da, or less than 700 Da, or less than 600 Da, or less than 500 Da, or less than 400 Da, or less than 300 Da, and even lower. For example, suitable thiolactones may have a molecular weigh of between 150 and 300 Da, or between 250 and 400 Da, or between 350 and 600 Da, or between 500 and 750 Da, or between 700 and 1,000 Da.

Referring back to the primary amine utilized to form the intermediate, there is a wide variety of possible primary amines that can be used, depending on the design constraints of the desired application of the nitric oxide precursors. The primary amine may include a cysteine or derivative thereof, a lysine or derivative thereof, a butylamine or derivative thereof, or a combination thereof. In embodiments when the cysteine or derivative thereof is utilized, the cysteine or derivative thereof may comprise cysteine, glutathione, acetyl cysteine, penicillamine, acetylpenicillamine, S-nitroso-n-acetylpenicillamine, bucillamine, or combinations thereof. Non-limiting examples of suitable cysteines or derivatives thereof are described in a journal article titled “S-Nitrosothiol Detection via Amperometric Nitric Oxide Sensor with Surface Modified Hydrogel Layer Containing Immobilized Organoselenium Catalyst” cited as Langmuir 2006, 22, 25, 10830-10836, which is incorporated by reference in its entirety. In most embodiments, contemplated primary amines will have a molecular weight of less than 1,200 Da, or less than 1,000 Da, or less than 750 Da, or less than 500 Da, or less than 400 Da, or less than 300 Da, or less than 200 Da, or less than 150 Da. For example, suitable primary amines may have a molecular weight of between 120 and 300 Da, or between 250 and 400 Da, or between 350 and 600 Da, or between 500 and 750 Da, or between 700 and 1,000 Da.

Referring back to the nitrosating compound utilized to form the nitric oxide precursor, the nitrosating compound may be any compound serving as a source of nitroso groups and will in general be a compound of formula NOX where X is an organic or inorganic anion or a group OR2 where R2 is an organic group. X may thus be an organic anion derived from a carboxylic acid, e.g., an alkane carboxylic acid containing 2-7 carbon atoms, nitrosating agents of this type including acetyl nitrite and propionyl nitrite. Where X is an inorganic anion this may be derived from, for example, a mineral acid, e.g., a halide ion such as chloride or bromide or a sulphate ion, or from a Lewis acid, e.g., a borofluoride ion. Other inorganic anions include hydroxide and sulphonate. Nitrosating compounds of this type thus include nitrosyl chloride, nitrosyl sulphate, nitrosyl borofluoride, nitrous acid and Fremys salt (potassium nitrosyldisulphonate). Where X is a group of formula OR2 the organic group R2 may be, for example, a lower alkyl group, such as containing 1-9 carbon atoms, e.g., ethyl, n-propyl, isopropyl, n-butyl, t-butyl or isopentyl.

In certain embodiments, the nitrosating compound comprises a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, nitrite salts, ion paired nitrite, silver nitrite, zinc nitrite, iron nitrite, copper nitrite, transition metal-nitrite compounds, or combinations thereof. Also, nitric oxide gas may be used as a nitrosating agent.

In various embodiments, the nitrosating compound has a weight average molecular weight of no greater than 10,000 g/mol, alternatively no greater than 1,000 g/mol, alternatively no greater than 500 g/mol, or alternatively no greater than 250 g/mol. From a different perspective, the nitrosating compound may have a weight average molecular weight of from about 10 g/mol to about 10,000 g/mol, alternatively from about 10 g/mol to about 1,000 g/mol, alternatively from about 10 g/mol to about 500 g/mol, or alternatively from about 10 g/mol to about 250 g/mol. For example, NaNO2 has a weight average molecular weight of 69 g/mol and butyl nitrite has a weight average molecular weight of 103 g/mol. Without being bound by theory, it is believed that reaction kinetics of forming the reaction product is improved by utilizing the nitrosating compounds having lower weight average molecular weights.

Therefore, it should be appreciated that the resultant nitric oxide precursor will have a relatively small molecular weight and as such will preferably not include a polymer with four or more than four repeat units. Therefore, in some embodiments the nitric oxide precursor will have no hetero- or homopolymer as a component. On the other hand, where the primary amine comprises a polypeptide, it is generally preferred that the polypeptide has less than ten amino acids, or less than eight, or less than six, or less than four amino acids.

As introduced above, it is generally preferred that the thiolactone and the primary amine will react in the presence of a solvent to form an intermediate, and the intermediate and the nitrosating compound will then react to form the nitric oxide precursor. If utilized, the solvent may be included in various amounts. The solvent may be aqueous, organic, non-organic, or combinations thereof. In certain embodiments, the solvent is an aqueous solvent, such as water or a mixture of water and methanol. In other embodiments, the solvent may comprise an organic solvent, such as tetrahydrofuran. Thus, most typically the solvent may be characterized as a polar or polar alcoholic solvent. Other non-limiting examples of suitable solvents include aromatics, aliphatics, ketones, such as methyl ethyl ketone, isobutyl ketone, ethyl amyl ketone, acetone, alcohols, such as methanol, ethanol n-butanol isopropanol esters, such as ethyl acetate, glycols, such as ethylene glycol propylene glycol ethers, such as tetrahydrofuran, ethylene glycol mono butyl ether, or combinations thereof. In further contemplated aspects, where the solvent is a mixture of an organic solvent with water, the solvent will have a relatively low water content such as equal or less than 25 vol %, or equal or less than 20 vol %, or equal or less than 15 vol %, or equal or less than 10 vol %, or equal or less than 5 vol %, or equal or less than 2.5 vol %.

The components utilized to form the nitric oxide precursor may be combined in a reaction mixture. It is to be appreciated that the components of the reaction mixture are not yet reacted with each other. The reaction mixture for forming the reaction product may comprise the thiolactone in an amount of from about 1 to about 80 wt. %, alternatively from about 1 to about 7 wt. %, or alternatively from about 50 to about 80 wt. %, based on a total weight of the reaction mixture. The reaction mixture for forming the reaction product may comprise the primary amine in an amount of from about 1 to about 80 wt. %, alternatively from about 1 to about 7 wt. %, or alternatively from about 50 to about 80 wt. %, based on a total weight of the reaction mixture. The reaction mixture for forming the reaction product may comprise the nitrosating compound in an amount of from about 1 to about 75 wt. %, alternatively from about 1 to about 10 wt. %, or alternatively from about 10 to about 75 wt. %, based on a total weight of the reaction mixture. When utilized, the reaction mixture for forming the reaction product may comprise the solvent in an amount of from about 1 to about 99 wt. % based on a total weight of the reaction mixture.

In various embodiments, the thiolactone and the primary amine are reacted in the presence of an acid. The acid may be utilized to improve formation and/or stability of the reaction product, such as when utilizing primary amine comprising amino acids, such as cysteine. In certain embodiments, the acid may comprise hydrochloric acid. Other non-limiting examples of suitable acids includes citric acid, methane sulfonic acid, ethane sulfonic acid, propane sulfonic acid, butane sulfonic acid, acetic acid, hydroxy acetic acid, propionic acid, hydroxy propionic acid, α-ketopropionic acid, butyric acid, mandelic acid, valeric acid, succinic acid, tartaric acid, malic acid, oxalic acid, fumaric acid, adipic acid, maleic acid, sorbic acid, benzoic acid, succinic acid, glutaric acid, adipic acid, α-hydroxy acids, ethylenediaminetetraacctic acid (EDTA), phosphonic acid, octyl phosphoric acid, acrylic acid, polyacrylic acid, aspartic acid, polyaspartic acid, p-hydroxybenzoic acids, iminoacetic acids, or combinations thereof. It is to be appreciated that the acid may be included as part of any component of the composition (e.g., carrier, solvent, etc.) or the reactants of the reaction product.

In other embodiments, the reaction product is formed substantially free of the presence of an acid. The inventors contemplate that the reaction product is substantially free of the acid. The phrase “substantially free” as used herein refers to either the complete absence of the acid or a minimal amount thereof merely as impurity, unintended byproduct of another ingredient, or in an amount that has a negligible impact on the composition or the nitric oxide precursor. In certain embodiments, “substantially free” means that the acid is present in the reaction product in an amount of less than 0.5 wt. %, less than 0.25 wt. %, less than 0.1 wt. %, less than 0.05 wt. %, or less than 0.01 wt. %, or even 0 wt. %, based on a total weight of the reaction product.

In some embodiments, the reaction mixture for forming the nitric oxide precursor may include a thiol-containing compound in addition to the thiolactone and the primary amine containing a thiol group. When utilized, there is a wide variety of possible thiol-containing compounds that can be used, depending on the design constraints of the desired application of the nitric oxide precursor. The thiol-containing compound can include, but is not limited to, one or more of 1,2-ethane dithiol, 2,3-dimercaptopropanol, pyrithione, dithioerythritol, 3,4-dimercaptotoluene, 2,3-butanedithiol, 1,3-propanedithiol, 2-hydroxypropane thiol, 1-mercapto-2-propanol, dithioerythritol and dithiothreitol. Other exemplary thiol-containing compounds include alpha-lipoic acid, methanethiol (CH3SH [m-mercaptan]), ethanethiol (C2H5SH [e-mercaptan]), 1-propanethiol (C3H7SH [n-P mercaptan]), 2-propanethiol (CH3CH(SH)CH3 [2C3 mercaptan]), butanethiol (C4H9SH ([n-butyl mercaptan]), tert-butyl mercaptan (C(CH3)3SH [t-butyl mercaptan]), pentanethiols (C5H11SH [pentyl mercaptan]), coenzyme A, lipoamide, glutathione, cysteine, cystine, 2-mercaptoethanol, dithiothreitol, dithioerythritol, 2-mercaptoindole, transglutaminase, (11-mercaptoundecyl)hexa(ethylene glycol), (11-mercaptoundecyl)tetra(ethylene glycol), (11-mercaptoundecyl)tetra(ethylene glycol) functionalized gold nanoparticles, 1,1′,4′,1″-terphenyl-4-thiol, 1,11-undecanedithiol, 1,16-hexadecanedithiol, 1,2-ethanedithiol, 1,3-propanedithiol, 1,4-benzenedimethanethiol, 1,4-butanedithiol, 1,4-butanedithiol diacetate, 1,5-pentanedithiol, 1,6-hexanedithiol, 1,8-octanedithiol, 1,9-nonanedithiol, adamantanethiol, 1-butanethiol, 1-decanethiol, 1-dodecanethiol, 1-heptanethiol, 1-heptanethiol purum, 1-hexadecanethiol, 1-hexanethiol, 1-mercapto-(triethylene glycol), 1-mercapto-(triethylene glycol) methyl ether functionalized gold nanoparticles, 1-mercapto-2-propanol, 1-nonanethiol, 1-octadecanethiol, 1-octanethiol, 1-octanethiol, 1-pentadecanethiol, 1-pentanethiol, 1-propanethiol, 1-tetradecanethiol, 1-tetradecanethiol purum, 1-undecanethiol, 11-(1H-pyrrol-1-yl)undecane-1-thiol, 11-amino-1-undecanethiol hydrochloride, 11-bromo-1-undecanethiol, 11-mercapto-1-undecanol, 11-mercapto-1-undecanol, 11-mercaptoundecanoic acid, 11-mercaptoundecanoic acid, 11-mercaptoundecyl trifluoroacetate, 11-mercaptoundecylphosphoric acid, 12-mercaptododecanoic acid, 12-mercaptododecanoic acid, 15-mercaptopentadecanoic acid, 16-mercaptohexadecanoic acid, 16-mercaptohexadecanoic acid, 1H,1H,2H,2H-perfluorodecanethiol, 2,2′-(ethylenedioxy)diethanethiol, 2,3-butanedithiol, 2-butanethiol, 2-ethylhexanethiol, 2-methyl-1-propanethiol, 2-methyl-2-propanethiol, 2-phenylethanethiol, 3,3,4,4,5,5,6,6,6-nonafluoro-1-hexanethiol purum, 3-(dimethoxymethylsilyl)-1-propanethiol, 3-chloro-1-propanethiol, 3-mercapto-1-propanol, 3-mercapto-2-butanol, 3-mercapto-N-nonylpropionamide, 3-mercaptopropionic acid, 3-mercaptopropyl-functionalized silica gel, 3-methyl-1-butanethiol, 4,4′-bis(mercaptomethyl)biphenyl, 4,4′-dimercaptostilbene, 4-(6-mercaptohexyloxy)benzyl alcohol, 4-cyano-1-butanethiol, 4-mercapto-1-butanol, 6-(ferrocenyl)hexanethiol, 6-mercapto-1-hexanol, 6-mercaptohexanoic acid, 8-mercapto-1-octanol, 8-mercaptooctanoic acid, 9-mercapto-1-nonanol, biphenyl-4,4′-dithiol, butyl 3-mercaptopropionate, copper(I) 1-butanethiolate, cyclohexanethiol, cyclopentanethiol, decanethiol functionalized silver nanoparticles, dodecanethiol functionalized gold nanoparticles, dodecanethiol functionalized silver nanoparticles, hexa(ethylene glycol)mono-11-(acetylthio)undecyl ether, mercaptosuccinic acid, methyl 3-mercaptopropionate, octanethiol functionalized gold nanoparticles, PEG dithiol, S-(11-bromoundecyl)thioacetate, S-(4-cyanobutyl)thioacetate, thiophenol, triethylene glycol mono-11-mercaptoundecyl ether, trimethylolpropane tris(3-mercaptopropionate), [11-(methylcarbonylthio)undecyl]tetra-(ethylene glycol), m-carborane-9-thiol, p-terphenyl-4,4″-dithiol, tert-dodecylmercaptan, or tert-nonyl mercaptan.

In certain embodiments, when utilized, the thiol-containing compound includes a thiol-derivatized polymer or filler. It is to be appreciated that the thiol-containing compound may be included as part of a peptide or other macromolecules so long as the thiol-containing compound is compatible with the components of the reaction mixture of the reaction product.

In various embodiments, when utilized, the thiol-containing compound has a weight average molecular weight of no greater than 500,000 g/mol, alternatively no greater than 100,000 g/mol, alternatively no greater than 10,000 g/mol, alternatively no greater than 1,000 g/mol, or alternatively no greater than 500 g/mol. From a different perspective, the thiol-containing compound may have a weight average molecular weight of from about 10 g/mol to about 500,000 g/mol, alternatively from about 10 g/mol to about 100,000 g/mol, alternatively from about 10 g/mol to about 1,000 g/mol, or alternatively from about 10 g/mol to about 500 g/mol.

In certain embodiments, the nitric oxide precursor is in a crystalline form. Thus, and viewed from a different perspective, the inventor contemplates that crystallization can pull the compound out of solution and thereby exclude ions that may cause decomposition. In contrast to crystallization, granular nitric oxide precursors may exhibit a greater amount of impurities as compared to crystallized nitric oxide precursors due to the trapping of contaminants. In various embodiments, crystallization includes a step of at least partially (e.g., at least 50%, or at least 60%, or at least 70%, or at least 80%, or at least 90%, or at least 95%, or at least 99%) removing the solvent after formation of the nitric oxide precursor. Crystallization may proceed via nucleation, supersaturation, and/or cooling. Methods of crystallization are well known to those of skill in the art and are described in textbooks such as A. Mersmann, Crystallization Technology Handbook (2001) CRC; 2nd ed. ISBN 0-8247-0528-9, which is incorporated by reference in its entirety.

In that context, it should be appreciated that a crystallized nitric oxide precursor may exhibit an improved storage stability as compared to a non-crystallized nitric oxide precursor. In various embodiments, the crystallized nitric oxide precursor exhibits minimal degradation at 4° C. for a time period of at least 1 month, alternatively at least 2 months, alternatively at least 3 months, alternatively at least 6 months, or alternatively at least 12 months. In other embodiments, the crystallized nitric oxide precursor exhibits minimal degradation at 23° C. for a time period of at least 1 month, alternatively at least 2 months, alternatively at least 3 months, alternatively at least 6 months, or alternatively at least 12 months. The phrase “minimal degradation” means that crystallized nitric oxide precursor exhibits a decrease in total weight over a desired time period of no greater than 10 wt. %, alternatively no greater than 5 wt. %, alternatively no greater than 4 wt. %, alternatively no greater than 3 wt. %, alternatively no greater than 2 wt. %, alternatively no greater than 1 wt. %, or alternatively no greater than 0.1 wt. %.

As introduced above, a composite article is also contemplated herein. The composite article includes a carrier and the nitric oxide precursor. In some embodiments, the composite article exhibits minimal release of nitric oxide for a time period of at least 5 minutes hours after forming the nitric oxide precursor. In various embodiments, the composite article exhibits minimal release of nitric oxide after forming the nitric oxide precursor for a time period of at least at least 10 minutes, alternatively at least 15 minutes, alternatively at least 30 minutes, alternatively at least 45 minutes, alternatively at least 1 hour, alternatively at least 2 hours, alternatively at least 3 hours, alternatively at least 4 hours, alternatively at least 5 hours, alternatively at least 6, alternatively at least 7 hours, alternatively at least 8 hours, alternatively at least 9 hours, alternatively at least 10 hours, alternatively at least 11 hours, alternatively at least 12, alternatively at least 13 hours, alternatively at least 14 hours, alternatively at least 15 hours, alternatively at least 16 hours, alternatively at least 17 hours, alternatively at least 18 hours, alternatively at least 19 hours, alternatively at least 20 hours, alternatively at least 21 hours, alternatively at least 22 hours, alternatively at least 23 hours, alternatively at least 24 hours, alternatively at least 48 hours, at least seven days, at least four weeks, at least 26 weeks, or alternatively at least 365 days, depending on the specific amine compound used and how the precursor is stored. The carrier may comprise a silica gel, a sodium polyacrylate, or a combination thereof. However, it is to be appreciated that any other carrier may be utilized.

Non-limiting examples of other suitable carriers include filter paper, polyacrylates, polyvinylchlorides, polydimethylsiloxanes, polyurethanes, and combinations thereof. Such carriers are well known to those of skill in the art and are described in textbooks such as Remington's Pharmaceutical Sciences, Mack Publishing Co., Easton, Pa., 1985, which is incorporated by reference in its entirety.

The composite article may comprise the nitric oxide precursor in an amount of from about 0.5 to about 10 wt. %, from about 10 to about 25 wt. %, from about 25 to about 50 wt. %, from about 50 to about 70 wt. %, or from about 70 to about 90 wt. %, such as, for example, from about 5 to about 25 wt. %, based on a total weight of the composite article. The composite article may comprise the carrier in an amount of from about 5 to about 95 wt. %, alternatively from about 10 to about 80 wt. %, or alternatively from about 80 to about 95 wt. %, based on a total weight of the composite article.

In exemplary embodiments, the nitric oxide precursor is included in a detergent composition or a cleaning composition. As used herein the phrases “detergent composition” or “cleaning composition” includes compositions and formulations designed for cleaning soiled material. Such compositions include, but are not limited to, object cleaning composition, medical device cleaning compositions, hard surface cleaning compositions, dishware cleaning compositions, laundry cleaning compositions and detergents, spray products, dry cleaning agent or composition, unit dose formulation, delayed delivery formulation, detergent contained on or in a porous substrate or nonwoven sheet, detergent contained on or in a water-soluble film, and other suitable forms that may be apparent to one skilled in the art in view of the teachings herein. The multi-component composition may have a form selected from liquid, powder, single-phase or multi-phase unit dose, 2-layer paper carrier, pouch, tablet, gel, paste, bar, or flake.

In these and other embodiments, the detergent or cleaning composition further comprises a cleaning agent. The cleaning agent comprises a detergent, an enzyme, or a combination thereof. A non-limiting example of a suitable cleaning agent include Alconox powder. In embodiments when the cleaning agent comprises a detergent, the detergent may comprise a surfactant, such as amine oxide, alkylbenzene sulfonate, alkyl ether sulfate, fatty alcohol ethoxylate, alkyl glycosides, alkoxylated fatty acid alkyl esters, amine oxides, fatty acid alkanolamides, hydroxy mixed ethers, sorbitan fatty acid esters, polyhydroxy fatty acid amides, and alkoxylated alcohols.

In embodiments when the cleaning agent comprises an enzyme, the enzyme may comprise one or more enzymes, which can display a catalytic activity in a detergent, such as a protease, amylase, lipase, cellulase, hemicellulase, mannanase, pectin-cleaving enzyme, tannase, xylanase, xanthanase, β-glucosidase, carrageenase, perhydrolase, oxidase, oxidoreductase, and mixtures thereof. In certain embodiments, the enzyme comprises proteases, amylases (e.g., α-amylases), cellulases, lipases, hemicellulases, pectinases, mannanases, β-glucanases, or combinations thereof. The properties of the enzyme should be compatible with the multi-component composition (i.e. pH-optimum, compatibility with other enzymatic and non-enzymatic ingredients, etc.). If utilized, the enzyme should be present in effective amounts.

When utilized, the protease may be of animal, vegetable or microbial origin, including chemically or genetically modified mutants. Microbial origin is preferred. It may be an alkaline protease, such as a serine protease or a metalloprotease. A serine protease may for example be of the Si family, such as trypsin, or the S8 family such as subtilisin. A metalloproteases protease may for example be a thermolysin from e.g., family M4, M5, M7 or M8.

When utilized, suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g. H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, WO 97/07202, WO 00/060063, WO2007/087508 and WO 2009/109500, which are incorporated by reference in their entirety.

When utilized, suitable amylases include those of bacterial or fungal origin.

Chemically modified or protein engineered mutants are included. Amylases include, for example, α-amylases obtained from Bacillus, e.g., a special strain of Bacillus lichenformis, described in more detail in GB 1,296,839. Examples of useful amylases are the variants described in WO 94/02597, WO 94/18314, WO 96/23873, and WO 97/43424, which are incorporated by reference in their entirety.

When utilized, suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757, and WO 89/09259, which are incorporated by reference in their entirety.

When utilized, suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257, which are incorporated by reference in their entirety.

The detergent or cleaning composition may further include additives, such as builders, bleaching agents, electrolytes, nonaqueous solvents, pH adjusting agents, fragrances, perfume carriers, fluorescent agents, dyes, hydrotropes, foam inhibitors, silicone oils, antiredeposition agents, graying inhibitors, shrinkage preventers, anti-creasing agents, dye transfer inhibitors, antimicrobial substances, germicides, fungicides, antioxidants, preservatives, corrosion inhibitors, antistatic agents, bitter agents, ironing aids, hydrophobizing and impregnating agents, swelling and anti-slip agents, softening components, and UV absorbers.

The detergent or cleaning composition may comprise the nitric oxide precursor in an amount of from about 0.01 to about 40 wt. %, alternatively from about 0.01 to about 5 wt. %, alternatively from about 5 to about 10 wt. %, or alternatively from about 10 to about 40 wt. %, based on a total weight of the composition. The detergent composition or a cleaning composition may comprise the cleaning agent in an amount of from about 1 to about 99 wt. % based on a total weight of the composition. The detergent composition or a cleaning composition may comprise the solvent in an amount of from about 1 to about 99 wt. % based on a total weight of the composition.

In various embodiments, the detergent or cleaning composition described herein can be filled into a water-soluble envelope and thus be part of a water-soluble package. The water-soluble envelope may be formed by a water-soluble film material. Such water-soluble packages can be produced either by vertical form fill seal (VFFS) methods or by thermoforming methods.

The envelope can be made of one layer or of two or more layers of the water-soluble film material. The water-soluble film material of the first layer and of the other layers, if present, can be the same or different. The water-soluble envelope, for example, is made from a water-soluble film material selected from the group comprising polymers or polymer mixtures. The water-soluble envelope may contain polyvinyl alcohol or a polyvinyl alcohol copolymer. Polymers selected from the group comprising acrylic acid-containing polymers, polyacrylamides, oxazoline polymers, polystyrene sulfonates, polyurethanes, polyesters, polyether polylactic acid, and/or mixtures of the above polymers, can be added to a film material that is suitable for producing the water-soluble envelope.

The thermoforming method generally includes forming a first layer from a water-soluble film material to create convexities for receiving a composition therein, filling the composition into the convexities, covering the convexities filled with the composition with a second layer of a water-soluble film material, and sealing the first and second layers together at least around the convexities. The water-soluble package comprising the liquid washing and the water-soluble envelope can have one or more chambers. The water-soluble packages can have a substantially dimensionally stable spherical and pillow-shaped configuration with a circular, elliptical, square, or rectangular basic form. The chambers may be isolated from one another.

A method of sterilizing or sanitizing an article is also provided herein. The method comprises applying the nitric oxide precursor described above to the article.

A method of forming the nitric oxide precursor is provided herein. The method comprises combining the thiolactone and the primary amine, optionally in the presence of a solvent and optionally in the presence of an acid, to form the intermediate. In various embodiments, the method may include allowing the intermediate to rest for a time period of from 1 minute to 24 hours, alternatively from 1 minute to 6 hours, alternatively from 10 minutes to 4 hours, or alternatively from 30 minutes to 150 minutes. The method further comprises combining the intermediate and the nitrosating compound to form the nitric oxide precursor.

In various embodiments, the steps of combining and the step of resting are performed at a temperature of from about 1° C. to about 25° C.

In exemplary embodiments, a thiolactone is reacted with a primary amine such as cysteine, penicillamine, glutathione, leucine, isoleucine, lysine, etc. to form the intermediate in a ring-opening reaction to so generate a (typically secondary or tertiary) thiol group. The physiochemical properties of reactivity, hydrophobicity, number of NO donors, stability of the NO donors, etc. can be tuned to create an array of properties. This allows precise tuning of NO loading, NO release, partition coefficient, etc. of the donors when incorporated into polymer matrices, powders, and solutions. To perform this reaction, the thiolactone and the primary amine compound are dissolved in a solvent and allowed to react. This reaction opens the thiolactone ring, forming an amide bond with the primary amine and exposes the thiol group(s) in the thiolactone. The thiol group(s) of the intermediate are then converted to the nitrosothiol by reacting the intermediate with a nitrosating agent such as sodium nitrite or tert-butylnitrite to form the nitric oxide precursor.

The formed nitric oxide precursors may exhibit a green or red color, depending on the molecular structure of the primary amines and the substitution around the thiol used. Advantageously, these nitric oxide precursors can be crystalized out of solution or used in solution with different matrices and/or carriers (e.g., filter paper, polyacrylates, PVC, PDMS, PU, silica gel powder, sodium polyacrylate, etc.). It should further be appreciated that the above noted reaction conditions for forming the nitric oxide precursors are mild as compared to conventional reaction products and methods for forming nitric oxide precursors. Notable, using the ring-opening reaction and subsequent nitrosation will allow the reaction to be performed at ambient temperature (e.g., between 15 and 25° C.), pressure (e.g., between 950 and 1,060 mbar), and humidity (e.g., between 15-90% relative humidity), and can be carried without protection from ambient light.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Example 1A: SNAP-Cysteine

29.8 mg of cysteine (primary amine) and 40 mg of N-(2,2-Dimethyl-4-oxo-3-thietanyl)acetamide (thiolactone) were mixed in an aqueous solvent solution of 1 mL of water, 4 mL of methanol and 100 μL 1M HCl to form the intermediate in solution. After resting for 1 hour, 50 mg of NaNO₂ (nitrosating compound) was added to the intermediate solution to form the nitric oxide precursor.

### Example 1B: SNAP-Leucine

31.4 mg of leucine (primary amine) and 40 mg of N-(2,2-Dimethyl-4-oxo-3-thietanyl)acetamide (thiolactone) were mixed in an aqueous solvent solution of 1 mL of water, 4 mL of methanol and 100 μL 1M HCl to form the intermediate solution. After resting for 1 hour, 50 mg of NaNO2 (nitrosating compound) was added to the intermediate solution to form the nitric oxide precursor.

### Example 1C: SNAP-Penicillamine

36 mg of penicillamine (primary amine) and 40 mg of N-(2,2-Dimethyl-4-oxo-3-thietanyl)acetamide (thiolactone) were mixed in an aqueous solvent solution of 1 mL of water, 4 mL of methanol and 100 μL 1M HCl to form the intermediate solution. After resting for 1 hour, 50 mg of NaNO2 (nitrosating compound) was added to the intermediate solution to form the nitric oxide precursor.

### Example 1D: SNAP-Lysine

23 mg of lysine (primary amine) and 40 mg of N-(2,2-Dimethyl-4-oxo-3-thietanyl)acetamide (thiolactone) were mixed in an aqueous solvent solution of 200 μL of water, 1.8 mL of methanol and 100 μL 1M HCl to form the intermediate solution. After resting for 1 hours, 50 mg of NaNO₂ (nitrosating compound) was added to the intermediate solution to form the nitric oxide precursor.

### Example 1E: SNAP-acetyl Lysine

29 mg of acetyllysine (primary amine) and 40 mg of N-(2,2-Dimethyl-4-oxo-3-thietanyl)acetamide (thiolactone) were mixed in an aqueous solvent solution of 200 μL of water, 1.8 mL of methanol and 100 μL 1M HCl to form the intermediate solution. After resting for 1 hour, 50 mg of NaNO2 (nitrosating compound) was added to the intermediate solution to form the nitric oxide precursor.

### Example 1A-E: Observations

After 1 hour, solutions turned deep red/green; green, darker green, deep red/green and deep red/green—as expected for the formation of SNAP and NOCys (green and red RSNOs), SNAP only (1 green RSNO), SNAP and nitrosopenicillamine (2 green RSNOs), SNAP only (1 green RSNO), and SNAP only (1 green RSNO).

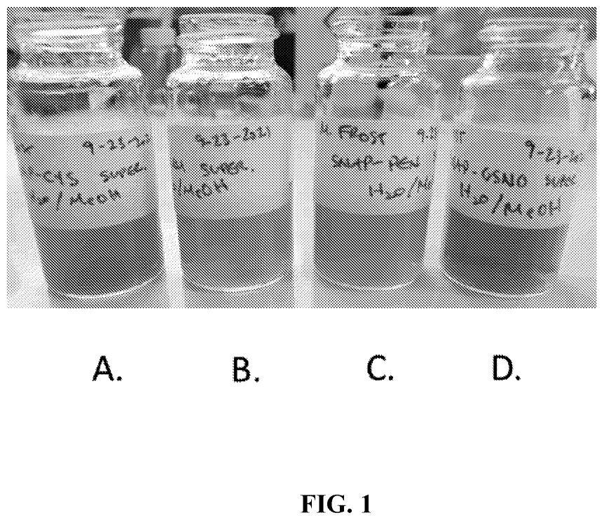

The inventor obtained highly concentrated solutions of same colors after evaporation of the methanol which is indicative of high stability. After 24 hours, the colors persisted which indicated high stability. Exemplary samples of the reaction products are shown in FIG. 1A-FIG. 1D.

### Example 2A: SNAP-Cysteine

28 mg of cysteine (primary amine) and 40.8 mg of N-(2,2-Dimethyl-4-oxo-3-thietanyl)acetamide (thiolactone) were mixed in a 5 mL of methanol to form the intermediate solution. After resting for 2 hours, 200 mg of tert-butylnitrite (nitrosating compound) was added to the intermediate solution to form the nitric oxide precursor.

### Example 2B: SNAP-Leucine

33 mg of leucine (primary amine) and 40.8 mg of N-(2,2-Dimethyl-4-oxo-3-thietanyl)acetamide (thiolactone) were mixed in a 5 mL of methanol to form the intermediate solution. After resting for 2 hours, 200 mg of tert-butylnitrite (nitrosating compound) was added to the intermediate solution to form the nitric oxide precursor.

### Example 2C: SNAP-Penicillamine

34.5 mg of penicillamine (primary amine) and 40.8 mg of N-(2,2-Dimethyl-4-oxo-3-thietanyl)acetamide (thiolactone) were mixed in a 5 mL of methanol to form the intermediate solution. After resting for 2 hours, 200 mg of tert-butylnitrite (nitrosating compound) was added to the intermediate solution to form the nitric oxide precursor.

### Example 2D: SNAP-Glutathione

76.6 mg of glutathione (primary amine) and 40.8 mg of N-(2,2-Dimethyl-4-oxo-3-thietanyl)acetamide (thiolactone) were mixed in a 5 mL of methanol to form the intermediate solution. After resting for 2 hours, 200 mg of tert-butylnitrite (nitrosating compound) was added to the intermediate solution to form the nitric oxide precursor.

### Example 2A-D: Observations

After 1 hour, solutions turned deep red/green; green, darker green, red/green—as expected for the formation of SNAP and NOCys (1 green and 1 red RSNOs), SNAP only (1 green RSNO), SNAP and nitrosopenicillamine (2 green RSNOs), SNAP-GSNO (1 green and 1 red RSNO).

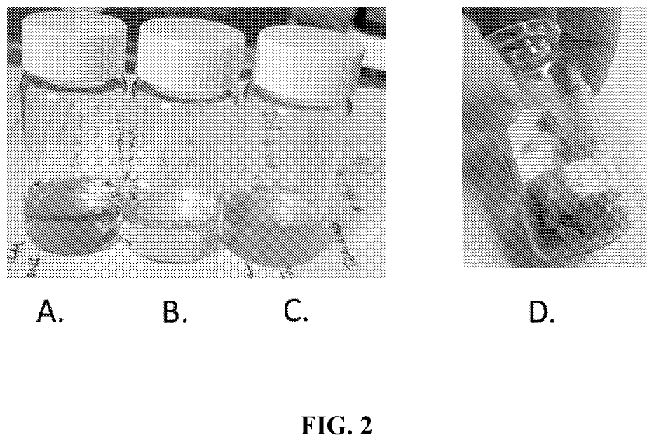

The inventor obtained highly concentrated solutions of same colors after evaporation of the methanol which is indicative of high stability. After 24 hours, the colors persisted which indicated high stability. See FIG. 2 which shows an example of SNAP-Pen, SNAP-Leu, and SNAP-GSNO (FIG. 2A, FIG. 2B, and FIG. 2C respectively). FIG. 2D is an image of SNAP-Pen synthesized in dichloromethane and crystalized with all solvent removed. The green-red color is indicative of a high concentration of tertiary RSNOs which are green-red biaxial crystals.

### Example 3A: SNAP-n-Butylamine (Acid-Free)

22 μl of n-butylamine (primary amine) and 40 mg of N-(2,2-Dimethyl-4-oxo-3-thietanyl)acetamide (thiolactone) were mixed in a solvent solution of 2 mL of THF/toluene to form the intermediate solution. 200 mg of tert-butylnitrite (nitrosating compound) was then added to the intermediate solution to form the nitric oxide precursor. A dark green solution resulted.

### Example 3B: SNAP-n-Butylamine (Acid)

22 μl of n-butylamine (primary amine) and 40 mg of N-(2,2-Dimethyl-4-oxo-3-thietanyl)acetamide (thiolactone) were mixed in a solvent solution of 2 mL of THF/toluene and 20 μL of dodecybenzenelsulfonic acid to form the intermediate solution. 200 mg of tert-butylnitrite (nitrosating compound) was then added to the intermediate solution to form the nitric oxide precursor. The addition of acid accelerated the rate of formation of the RSNO. A dark green solution resulted.

### Example 3C: SNAP-Sec-Butylamine (Acid-Free)

22 μl of sec-butylamine (primary amine) and 40 mg of N-(2,2-Dimethyl-4-oxo-3-thietanyl)acetamide (thiolactone) were mixed in a solvent solution of 2 mL of THF/toluene to form the intermediate solution. 200 mg of tert-butylnitrite (nitrosating compound) was then added to the intermediate solution to form the nitric oxide precursor. A dark green solution resulted.

### Example 3D: SNAP-Sec-Butylamine (Acid)

22 μl of sec-butylamine (primary amine) and 40 mg of N-(2,2-Dimethyl-4-oxo-3-thietanyl)acetamide (thiolactone) were mixed in a solvent solution of 2 mL of THF/toluene and 20 μL of dodecybenzenelsulfonic acid to form the intermediate solution. 200 mg of tert-butylnitrite (nitrosating compound) was then added to the intermediate solution to form the nitric oxide precursor. The addition of acid accelerated the rate of formation of the RSNO. A dark green solution resulted.

### Example 3E: SNAP-Tert-Butylamine (Acid-Free)

22 μl of tert-butylamine (primary amine) and 40 mg of N-(2,2-Dimethyl-4-oxo-3-thietanyl)acetamide (thiolactone) were mixed in a solvent solution of 2 mL of THF/toluene to form the intermediate solution. 200 mg of tert-butylnitrite (nitrosating compound) was then added to the intermediate solution to form the nitric oxide precursor. A dark green solution resulted.

### Example 3F: SNAP-Tert-Butylamine (Acid)

22 μl of tert-butylamine (primary amine) and 40 mg of N-(2,2-Dimethyl-4-oxo-3-thietanyl)acetamide (thiolactone) were mixed in a solvent solution of 2 mL of THF/toluene and 20 μL of dodecybenzenelsulfonic acid to form the intermediate solution. 200 mg of tert-butylnitrite (nitrosating compound) was then added to the intermediate solution to form the nitric oxide precursor. The addition of acid accelerated the rate of formation of the RSNO. A dark green solution resulted.

### Example 3A-F: Composite Articles

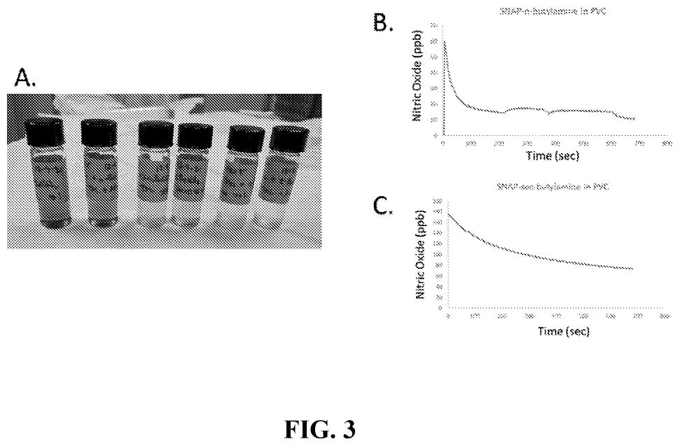

1 mL of each nitric oxide precursor solution was then combined with 1 mL of PVC polymer solution (0.1 g PVC/mL of THF) and cast into films. Green PVC polymers resulted that released NO. The same experiment was completed using the toluene as the solvent and RTV-3140 PDMS as the polymer. See FIG. 3A which shows the reaction mixtures immediately after the addition of tert-butyl nitrite when toluene was used as the solvent. See FIG. 3B and FIG. 3C which show NO release from 3-layer PVC films with SNAP-n-butylamine and SNAP-sec-butylamine, respectively with detection via chemiluminescence.

### Example 4: Composite Articles of SNAP-Lys Silica Gel and SNAP-Lys Sodium Polyacrylate

SNAP-Lys was synthesized from 23 mg of lysine, 40 mg of N-(2,2-Dimethyl-4-oxo-3-thietanyl)acetamide in 200 μL water and 1.8 mL methanol with 100 100 μL 1M HCl. After 1 hour, 50 mg NaNO₂ added. The solution was stored at 4° C. overnight and methanol was driven off with a stream of nitrogen.

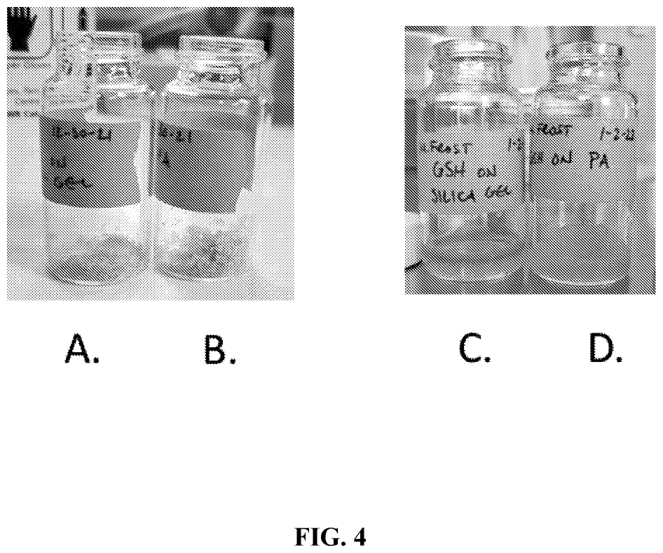

300 μL of the concentrated SNAP-Lys aqueous/methanol solution was then pipetted onto 500 mg of silica gel and 300 μL of the concentrated SNAP-Lys aqueous/methanol solution was pipetted onto 500 mg of sodium polyacrylate. Both sets of particles were allowed to air dry. Deep green-red desiccants resulted that were able to release NO. The same desiccants were also made that used GSNO as the NO source by combining 50 mg of GSH and 100 mg NaNO2 in 3 mL of H₂O. 1.5 mL of this red solution were then pipetted onto 500 mg of silica gel and 500 mg of sodium polyacrylate. A pink NO releasing desiccant resulted. See FIG. 4 for images of these NO releasing desiccants from SNAP-Lys (green; FIG. 4A and FIG. 4B) and GSNO (red; FIG. 4C and FIG. 4D), respectively.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

It must also be noted that, as used in the specification and the appended claims, the singular form “a,” “an,” and “the” comprise plural referents unless the context clearly indicates otherwise. For example, reference to a component in the singular is intended to comprise a plurality of components.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

Except in the examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately”, when referring to a specified, measurable value (such as a parameter, an amount, a temporal duration, and the like), is meant to encompass the specified value and variations of and from the specified value, such as variations of +/−10% or less, alternatively+/−5% or less, alternatively+/−1% or less, alternatively+/−0.1% or less of and from the specified value, insofar as such variations are appropriate to perform in the disclosed embodiments. Thus, the value to which the modifier “about” or “approximately” refers is itself also specifically disclosed.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A method of producing a nitric oxide precursor-containing consumer article, comprising:
reacting a thiolactone with a primary amine in a ring-opening reaction in a solvent to form an intermediate with a thiol group;
reacting the thiol group of the intermediate with a nitrosating compound in the solvent to thereby form a nitric oxide precursor having a nitrosothiol; and
treating the consumer article with the nitric oxide precursor having the nitrosothiol, and optionally at least partially removing the solvent;
wherein the consumer article is a desiccant, a detergent or cleaning solution, a detergent or cleaning powder, or a liner for sanitizing, wherein the nitric oxide precursor is capable of decomposing to form nitric oxide within 1 hour,
wherein the nitric oxide precursor exhibits minimal degradation for a time period of at least 1 month.

2. The method of claim 1, wherein the thiolactone has a molecular weight of less than 500 Da, and/or wherein the primary amine has a molecular weight of less than 500 Da.

3. The method of claim 1, wherein the thiolactone has a structure according to the following formula (III): 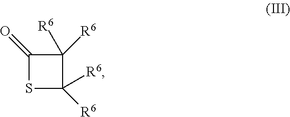
wherein each occurrence of R⁶ is independently a hydrogen, a hydroxyl, a substituted or unsubstituted C₁-C₆ alkyl, substituted or unsubstituted C₁-C₆ heteroalkyl, a substituted or unsubstituted C₂-C₆ alkenyl, a substituted or unsubstituted C₂-C₆ herteroalkenyl, a substituted or unsubstituted C₁-C₆ alkoxy, or a substituted or unsubstituted C₁-C₆ heteroalkoxy; and wherein when R6 is substituted, the substituent is selected for the group consisting of OH, SH, NH₃, NO₂, and halogen.

4. The method of claim 1, wherein the thiolactone comprises an amine group, and optionally wherein the thiolactone is N-(2,2-Dimethyl-4-oxo-3-thietanyl)-acetamide, N-acetylcysteine thiolactone, N-acetyl-homocysteine thiolactone, homocysteine thiolactone, or butyryl-homocysteine thiolactone.

5. (canceled)

6. (canceled)

7. The method of claim 1, wherein the primary amine is an amino acid, or wherein the primary amine is butylamine, cysteine, glutathione, acetyl cysteine, penicillamine, acetylpenicillamine, S-nitroso-n-acetylpenicillamine, or bucillamine.

8. (canceled)

9. The method of claim 1, wherein the nitrosating compound is an organonitrite or a nitrite salt, or wherein the nitrosating compound is sodium nitrite, calcium nitrite, potassium nitrite, tetrabutylammonium nitrite, dicyclohexylammonium nitrite, butylnitrite, isobutylnitrile, t-butylnitrite, amylnitrite, or pentylnitrite.

10. (canceled)

11. The method of claim 1, wherein the solvent is an aqueous solvent, and/or wherein the solvent comprises a polar alcoholic solvent.

12. (canceled)

13. (canceled)

14. The method of claim 1, wherein the steps of reacting are performed in the presence of an acid.

15. The method of claim 1, wherein the steps of reacting are performed at ambient temperature and pressure.

16. The method of claim 1, wherein step of at least partially removing the solvent removes at least 50% of the solvent.

17. (canceled)

18. The method of claim 1, further comprising a step of crystallizing the nitric oxide precursor.

19. The method of claim 1, wherein the nitric oxide precursor does not comprise a polymer having 4 or more repeat units.

20. The method of claim 1, wherein the step of treating comprises coating the consumer article with the nitric oxide precursor or blending the nitric oxide precursor as a solid with the consumer article.

21. A consumer article comprising a nitric oxide precursor for providing nitric oxide, comprising:
a reaction product of:
a thiolactone,
a primary amine, and
a nitrosating compound;
wherein the nitric oxide precursor is capable of decomposing to form nitric oxide within 1 hour;
wherein the nitric oxide precursor exhibits minimal degradation for a time period of at least 1 month; and
wherein the consumer article is treated with the nitric oxide precursor having the nitrosothiol, and wherein the consumer article is a desiccant, a detergent or cleaning solution, a detergent or cleaning powder, or a liner for sanitizing.

22. (canceled)

23. (canceled)

24. (canceled)

25. The consumer article of claim 21, wherein the nitric oxide precursor comprises a nitrosothiol, and wherein the nitrosothiol is capable of decomposing to form the nitric oxide, and/or wherein the nitric oxide precursor exhibits minimal decomposition to nitrogen oxide for at least 24 hours after forming the nitrogen oxide precursor.

26. (canceled)

27. The consumer article of claim 21, wherein the thiolactone is an amine-containing thiolactone.

28. (canceled)

29. The consumer article of claim 21, wherein the primary amine comprises a cysteine or derivative thereof, a lysine or derivative thereof, a butylamine or derivative thereof, or a combination thereof, and/or wherein the primary amine contains a thiol-functional group.

30. (canceled)

31. The consumer article of claim 21, wherein the primary amine comprises cysteine or derivative thereof, and wherein the cysteine or derivative thereof comprises cysteine, glutathione, acetyl cysteine, penicillamine, acetylpenicillamine, S-nitroso-n-acetylpenicillamine, bucillamine, or combinations thereof.

32. (canceled)

33. (canceled)

34. The consumer article of claim 21, wherein the nitric oxide precursor is in a crystalline form, and wherein the crystallized nitric oxide precursor exhibits improved storage stability as compared to the corresponding non-crystallized nitric oxide precursor.

35. A composite article comprising:
a carrier; and
a nitric oxide precursor that is a reaction product of a thiolactone, a primary amine, and a nitrosating compound;
wherein the nitric oxide precursor is coated onto the carrier, or blended as a solid with the carrier; and
wherein the composite article exhibits minimal release of nitric oxide for at least 1 month after forming the nitric oxide precursor; and
wherein, upon the release of nitric oxide, the nitric oxide precursor decomposes to nitric oxide within 1 hour.

36. (canceled)
